(19) United States
(12) Patent Application Publication
(10) Pub. No.: US 2022/0409578 A1
(43) Pub. Date: Dec. 29, 2022

(54) FUNCTIONALIZED 1,3-BENZENE DIOLS AND THEIR METHOD OF USE FOR THE TREATMENT OF RADIATION DERMATITIS AND OTHER SKIN DISORDERS

(71) Applicant: KANNALIFE SCIENCES, INC., Doylestown, PA (US)

(72) Inventors: Douglas BRENNEMAN, North Wales, PA (US); William A. KINNEY, Newtown, PA (US); Mark E. MCDONNELL, Lansdale, PA (US); Dean PETKANAS, Lloyd Harbor, NY (US)

(21) Appl. No.: 17/776,707

(22) PCT Filed: Nov. 13, 2020
(86) PCT No.: PCT/US2020/060587

§ 371 Date: May 13, 2022

Related U.S. Application Data
(60) Provisional application No. 62/934,861, filed on Nov. 13, 2019.

(51) Int. Cl. A61K 31/397; A61K 9/00; A61K 31/4192; A61P 17/18; A61P 29/00; A61K 47/24

(52) U.S. Cl. CPC A61K 31/397; A61K 9/0014; A61K 31/4192; A61P 17/18; A61P 29/00; A61K 47/24

(57) ABSTRACT

Methods, compounds and compositions for treating and preventing dermatological disorders and ocular irritancy are disclosed. The compounds and compositions comprise a functionalized 1,3-benzene diol, such as 5-(2-(1H-1,2,3-triazol-1-yl)ethyl)-2-((1R,6R)-3-methyl-6-(prop-1-en-2-yl)cyclohex-2-enyl)benzene-1,3-diol and ethyl 3-(3,5-dihydroxy-4-((1R,6R)-3-methyl-6-(prop-1-en-2-yl)cyclohex-2-enyl)benzyl)azetidine-1-carboxylate.

### BACKGROUND OF THE INVENTION

### 1. Field of Invention

This invention relates to dermatological treatment methods and compositions, and more particularly to the use of 1,3-benzene diols in such methods and compositions.

### 2. Description of Related Art

An emerging concept is that protection from free radicals produced by radiation therapy (RT) will protect skin tissue from damage and alleviate pain. See, e.g., Amber et al. (2014). Compounds capable of acting as protective agents can act by blocking damage caused by oxidative stress which is produced by radiation-mediated free radical attack of cellular components.

The major side effect of RT is skin tissue damage also known as radiodermatitis which occurs in 95% of cancer patients receiving RT. While acute inflammation is observed within hours of radiation treatment, this painful and common side effect develops over weeks and its severity with time progresses to erythema, dry or wet desquamation or ulceration (Perraud et al., 2019).

Currently no single agent has been shown to completely alleviate the radiation dermatitis side effect. Current therapies that focus on the symptoms include corticosteroid cream, antibiotics, zinc and amifostine. Because of the widespread incidence and the paucity of completely effective therapies, there is an urgent need for new and effective therapies, particularly those employing small molecules that are directed on regulating free radicals and decreasing the concentration of mediators of inflammation which are largely cytokines.

Allergic contact dermatitis to nickel ions (Ni⁺²) is an inflammatory dermatosis that is the most frequent cause of contact hypersensitivity in the industrialized world. Previous studies have shown that Ni⁺² ions trigger an inflammatory response by directly activating the Toll-like receptor 4 (Schmidt et al., 2010). Among the proinflammatory cytokines induced by Ni⁺² ions are TNFα, IL-8 and Il-1β.

1,3-benzene diols have been shown to prevent oxidative stress and nerve cell damage in cultures of the central and peripheral nervous systems (Brenneman et al., 2018; Brenneman et al., 2019; U.S. Pat. No. 9,611,213 B2; and U.S. Ser. No. 10/004,722 B2). These protective effects from increased free radicals and excessive calcium overload mediated by ethanol or paclitaxel could be prevented by an inhibitor of the sodium-calcium exchanger-1. In confirmative studies, a decrease in the gene expression of the mitochondrial sodium-calcium exchanger-1 with siRNA resulted in an inhibition of the protective effect of a 1,3-benzene diol. These studies support the involvement of this protective mechanism that is established for hippocampal and dorsal root ganglion neurons that may be pertinent to the regulation of oxidative stress in other tissues, including skin. Indeed, previous studies in keratinocytes have demonstrated that UV-B radiation causes an increase in intracellular calcium that is important for the release of the proinflammatory cytokine IL-1β (Feldmeyer et al., 2007).

Skin aging and oxidative stress is affected by aging that produces inflammation, disruption of the skin barrier, decreased ability to repair wounds and increased skin cancer. Most of the events that contribute to aging or radiation such as mitochondrial dysfunction, inflammation and abnormalities of extracellular matrix homeostasis are a consequence to oxidative stress (Kammeyer and Leuiten, 2015).

Accordingly, it is desired to provide new methods and compositions for treating or preventing dermatological disorders. It is further desired to provide new methods and compositions for treating or preventing skin disorders associated with radiation therapy. It is still further desired to provide new methods and compositions for treating or preventing allergic contact dermatitis.

All references cited herein are incorporated herein by reference in their entireties.

### BRIEF SUMMARY OF THE INVENTION

Accordingly, a first aspect of the invention comprises a functionalized 1,3-benzene diol selected from the group consisting of 5-(2-(1H-1,2,3-triazol-1-yl)ethyl)-2-((1R,6R)-3-methyl-6-(prop-1-en-2-yl)cyclohex-2-enyl)benzene-1,3-diol and ethyl 3-(3,5-dihydroxy-4-((1R,6R)-3-methyl-6-(prop-1-en-2-yl)cyclohex-2-enyl)benzyl)azetidine-1-carboxylate.

A second aspect of the invention comprises a method of treating or preventing a dermatological disorder, said method comprising applying to skin a therapeutically effective amount of a functionalized 1,3-benzene diol of formula (I), (II), (III), (IV), (V), (VI), (VII), (VIII), (IX), (X), (XI) or (XII) as described below.

In certain embodiments, the dermatological disorder is radiation dermatitis or allergic contact dermatitis.

In certain embodiments, the functionalized 1,3-benzene diol is 5-(2-(1H-1,2,3-triazol-1-yl)ethyl)-2-((1R,6R)-3-methyl-6-(prop-1-en-2-yl)cyclohex-2-enyl)benzene-1,3-diol.

In certain embodiments, the functionalized 1,3-benzene diol produces an analgesic effect.

In certain embodiments, the functionalized 1,3-benzene diol exhibits therapeutic effects by locally restricted tissue actions that do not involve significant entry in systemic circulation.

In certain embodiments, the method further comprises applying to the skin an additional agent therapeutically effective to treat irradiated skin in a formulation that both solubilizes and enhances protection against oxidative damage in skin tissue.

In certain embodiments, the method further comprises applying soy lecithin to the skin.

In certain embodiments, the soy lecithin comprises phosphatidylcholine with a polyunsaturation of the fatty acyl composition of at least 63%.

In certain embodiments, the method inhibits a release of inflammatory cytokines in skin tissue produced by irradiation.

In certain embodiments, the functionalized 1,3-benzene diol is applied to the skin at a concentration less than 3% (wt/vol) which does not produce skin cell irritancy at therapeutic doses.

A third aspect of the invention comprises a method of treating or preventing ocular irritation, said method comprising applying to ocular tissue a therapeutically effective amount of a functionalized 1,3-benzene diol, wherein the therapeutically effective amount does not produce ocular irritancy.

In certain embodiments of the method of treating or preventing ocular irritation the functionalized 1,3-benzene diol is 5-(2-(1H-1,2,3-triazol-1-yl)ethyl)-2-((1R,6R)-3-methyl-6-(prop-1-en-2-yl)cyclohex-2-enyl)benzene-1,3-diol.

A fourth aspect of the invention comprises a functionalized 1,3-benzene diol for use in treating or preventing a dermatological disorder. The functionalized 1,3-benzene diol is selected from the group consisting of formula (I), (II), (III), (IV), (V), (VI), (VII), (VIII), (IX), (X), (XI) and (XII) as described below.

A fifth aspect of the invention comprises a functionalized 1,3-benzene diol for use in treating or preventing ocular irritation. The functionalized 1,3-benzene diol is selected from the group consisting of formula (I), (II), (III), (IV), (V), (VI), (VII), (VIII), (IX), (X), (XI) and (XII) as described below.

### BRIEF DESCRIPTION OF SEVERAL VIEWS OF THE DRAWINGS

The invention will be described in conjunction with the following drawings, wherein:

FIG. 1 shows the decreased release of tumor necrosis factor alpha (TNF-α) into the culture medium of human epidermal keratinocytes irradiated with ultraviolet-B after treatment with various concentration of KLS-13022.

FIG. 2 shows the decreased release of interleukin-1p into the culture medium of human epidermal keratinocytes irradiated with ultraviolet-B after treatment with various concentration of KLS-13022.

FIG. 3 shows the decreased release of the chemokine CXCL5 into the culture medium of human epidermal keratinocytes irradiated with ultraviolet-B after treatment with various concentration of KLS-13022.

FIG. 4 shows the decreased release of interleukin-6 into the culture medium of ultraviolet-A-induced photoaging in human dermal fibroblast cells after treatment with KLS-13022.

FIG. 5 shows the decreased release of interleukin-8 into the culture medium of nickel-induced allergy in human dermal microvascular endothelial cells after treatment with various concentration of KLS-13022.

FIG. 6 shows the cell viability of human epidermal keratinocytes after treatment with KLS-13022.

FIG. 7 shows the cell viability of human dermal microvascular endothelial cells after treatment with KLS-13022.

FIG. 8 shows the cell viability of human dermal fibroblast cells after treatment with KLS-13022

FIG. 9 shows the testing of KLS-13022 on potential skin irritation activity as determined with the human skin-like EPIDERM in vitro model.

FIG. 10 shows the testing of KLS-13022 on the in vitro eye irritation activity as determined in the reconstructed human cornea-like model-EPIOCULAR.

FIG. 11 shows the determination of antioxidant capacity of KLS-13022.

FIG. 12 shows the Franz cell dermal penetration of formulation containing KLS-13022.

### DETAILED DESCRIPTION OF PREFERRED EMBODIMENTS OF THE INVENTION

### Glossary

As used herein, the term “halogen” shall mean chlorine, bromine, fluorine and iodine.

As used herein, unless otherwise noted, “alkyl” and/or “aliphatic” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆alkyl)₂amino, the alkyl groups may be the same or different.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Nonlimiting examples of alkenyl groups include ethenyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-ynyl (also propargyl), propyn-1-yl, and 2-methyl-hex-4-yn-1-yl. Nonlimiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Cycloalkyl rings can be optionally substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

The term “aryl,” wherein used alone or as part of another group, is defined herein as an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “arylalkyl” and “aralkyl” refer to the group -alkyl-aryl, where the alkyl and aryl groups are as defined herein. Aralkyl groups of the invention are optionally substituted. Examples of arylalkyl groups include, for example, benzyl, 1-phenylethyl, 2-phenylethyl, 3-phenylpropyl, 2-phenylpropyl, fluorenylmethyl and the like.

The terms “heterocyclic” and “heterocycle” and “heterocylyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the invention fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

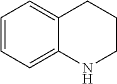

is, for the purposes of the invention, considered a heterocyclic unit. 6,7-Dihydro-5H-cyclopentapyrimidine having the formula:

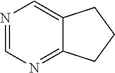

is, for the purposes of the invention, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

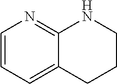

is, for the purposes of the invention, considered a heteroaryl unit.

Whenever a term or either of their prefix roots appear in a name of a substituent, the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated.

The following are non-limiting examples of substituents which can substitute for hydrogen atoms on a moiety: halogen (chlorine (Cl), bromine (Br), fluorine (F) and iodine(I)), —CN, —NO₂, oxo (═O), —OR¹¹, —SR¹¹, —N(R¹¹)₂, —NR¹¹C(O)R¹¹, —SO₂R¹¹, —SO₂OR¹¹, —SO₂N(R¹¹)₂, —C(O)R¹¹, —C(O)OR¹¹, —C(O)N(R¹¹)₂, C₁₋₆ alkyl, C₁₋₆ haloalkyl, C₁₋₆ alkoxy, C₂₋₈ alkenyl, C₂₋₈ alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, oxo, and R¹¹; wherein R¹¹, at each occurrence, independently is hydrogen, —OR¹², —SR¹², —C(O)R¹², —C(O)OR¹², —C(O)N(R¹²)₂, —SO₂R¹², —S(O)₂OR¹², —N(R¹²)₂, —NR¹²C(O)R¹², C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R¹¹ units taken together with the atom(s) to which they are bound form an optionally substituted carbocycle or heterocycle wherein said carbocycle or heterocycle has 3 to 7 ring atoms; wherein R¹², at each occurrence, independently is hydrogen, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R² units taken together with the atom(s) to which they are bound form an optionally substituted carbocycle or heterocycle wherein said carbocycle or heterocycle preferably has 3 to 7 ring atoms.

In certain embodiments, the substituents are selected from the group consisting of:

i) —OR¹³; for example, —OH, —OCH₃, —OCH₂CH₃, —OCH₂CH₂CH₃;
ii) —C(O)R¹³; for example, —COCH₃, —COCH₂CH₃, —COCH₂CH₂CH₃;
iii) —C(O)OR¹³; for example, —CO₂CH₃, —CO₂CH₂CH₃, —CO₂CH₂CH₂CH₃;
iv) —C(O)N(R¹³)₂; for example, —CONH₂, —CONHCH₃, —CON(CH₃)₂;
v) —N(R¹³)₂; for example, —NH₂, —NHCH₃, —N(CH₃)₂, —NH(CH₂CH₃);
vi) halogen: —F, —Cl, —Br, and —I;
vii) —CHₑX_{g}; wherein X is halogen, m is from 0 to 2, e+g=3; for example, —CH₂F, —CHF₂, —CF₃, —CCl₃, or —CBr₃;
viii) —SO₂R¹³; for example, —SO₂H; —SO₂CH₃; —SO₂C₆H₅;
ix) C₁-C₆ linear, branched, or cyclic alkyl;

### x) Cyano

### xi) Nitro;

xii) N(R¹³)C(O)R¹³;
xiii) Oxo (═O);
xiv) Heterocycle; and

### xv) Heteroaryl,

wherein each R¹³ is independently hydrogen, optionally substituted C₁-C₆ linear or branched alkyl (e.g., optionally substituted C₁-C₄ linear or branched alkyl), or optionally substituted C₃-C₆ cycloalkyl (e.g optionally substituted C₃-C₄ cycloalkyl); or two R¹³ units can be taken together to form a ring comprising 3-7 ring atoms. In certain aspects, each R¹³ is independently hydrogen, C₁-C₆ linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆, alkyl.

In all of the embodiments provided herein, examples of suitable optional substituents are not intended to limit the scope of the claimed invention. The compounds of the invention may contain any of the substituents, or combinations of substituents, provided herein.

For the purposes of the invention the terms “compound,” “analog,” and “composition of matter” stand equally well for the novel functionalized 1,3-benzenediols described herein, including all enantiomeric forms, diastereomeric forms, salts, and the like, and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence (e.g., in N(R¹²)₂, each R¹² may be the same or different than the other). Combinations of substituents and/or variables are permissible only if such combinations result in stable compounds.

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

Compounds of the Invention

The invention further comprises functionalized 1,3-benzenediols effective for treating or preventing dermatological disorders. The 1,3-benzenediols of the invention are preferably compounds of formula (I):

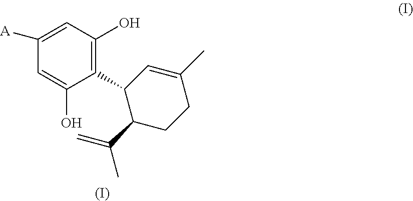

including hydrates, solvates, pharmaceutically acceptable salts, prodrugs and complexes thereof, wherein:
A is selected from the group consisting of

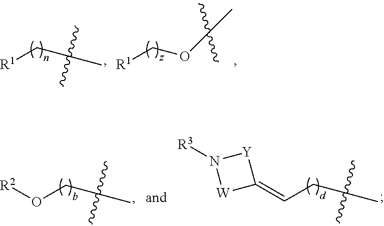

z is 0, 1, or 2;
when A is

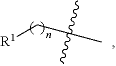

R¹ is selected from the group consisting of

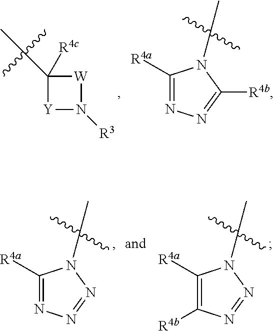

when A is

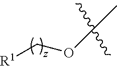

### and z is 0, R¹ is

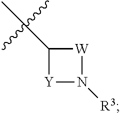

when A is

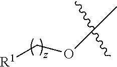

### and z is 1, R¹ is

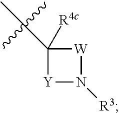

when A is

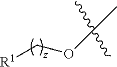

and z is 2, R¹ is selected from the group consisting of

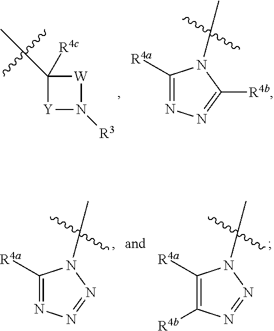

when R¹ is

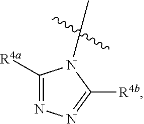

n is not 0;
when R¹ is

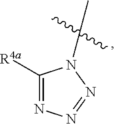

n is not 0;
when R¹ is

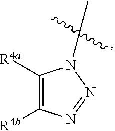

n is not 0;

### R² is

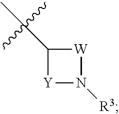

W is (CH₂)ₘ;
m is 1 or 2;
Y is (CH₂)_{q};
q is 1 or 2;
n is 0, 1, 2, or 3;
b is 0, 1, 2, or 3;
d is 0, 1, 2, or 3;
R³ is selected from the group consisting of COR⁵, CO₂R⁶, CONR^{7a}R^{7b}, SO₂NR^{7a}R^{7b}, SO₂R⁸, and optionally substituted heteroaryl;
R^{4a} and R^{4b} are each independently selected from the group consisting of hydrogen and C₁₋₆ alkyl;
R^{4c} is selected from the group consisting of hydrogen and OH;
R⁵ is selected from the group consisting of hydrogen, unsubstituted C₁₋₆ alkyl, substituted C₁₋₆ alkyl, unsubstituted heteroaryl, substituted heteroaryl, —C(R^{9a}R^{9b})NR^{7a}R^{7b}, and —C(R^{9a}R^{9b})OR¹⁰;
R⁶ is unsubstituted C₁₋₆ alkyl or substituted C₁₋₆ alkyl;
R^{7a} and R^{7b} are each independently selected from the group consisting of hydrogen, unsubstituted C₁₋₆ alkyl and substituted C₁₋₆ alkyl;
R⁸ is selected from the group consisting of hydrogen, unsubstituted C₁₋₆ alkyl, substituted C₁₋₆ alkyl, unsubstituted heteroaryl and substituted heteroaryl;
R^{9a} and R^{9b} are each independently selected from the group consisting of hydrogen, C₁₋₆ alkyl, C₃₋₇ branched alkyl, CH₂OH, CH(OH)CH₃, CH₂Ph, CH₂(4-OH-Ph), (CH₂)₄NH₂, (CH₂)₃NHC(NH₂)NH, CH₂(3-indole), CH₂(5-imidazole), CH₂CO₂H, CH₂CH₂CO₂H, CH₂CONH₂, and CH₂CH₂CONH₂; and
R¹⁰ is selected from the group consisting of hydrogen and C₁₋₆ alkyl.

The compounds of the invention further include enantiomers of compounds of the formula (I).

The compounds of the invention further include compounds of the formula (I) that are isotopically labeled with 1 to 10 deuterium atoms.

The compounds of the invention further include compounds having formula (II):

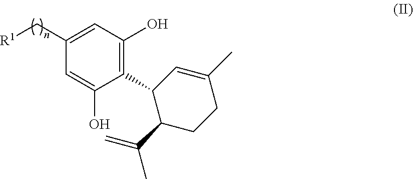

including hydrates, solvates, pharmaceutically acceptable salts, and complexes thereof, wherein R¹ and n of formula (II) are as defined above with respect to formula (I). In certain embodiments of the invention, R¹ and n of formula (II) are as defined in Table 1 below.

TABLE 1

| Entry | R¹ | n |
|---|---|---|
| 1 | 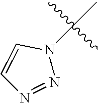 [SMILES: *n1ccnn1] | 1 |
| 2 | 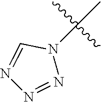 [SMILES: *n1cnnn1] | 1 |
| 3 | 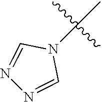 [SMILES: *n1cnnc1] | 1 |
| 4 | 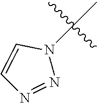 [SMILES: *n1ccnn1] | 2 |
| 5 | 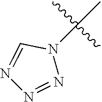 [SMILES: *n1cnnn1] | 2 |
| 6 | 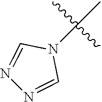 [SMILES: *n1cnnc1] | 2 |

The compounds of the invention further include enantiomers of compounds of the formula (II).

The compounds of the invention further include compounds of the formula (II) that are isotopically labeled with 1 to 10 deuterium atoms.

The compounds of the invention further include compounds having formula (III):

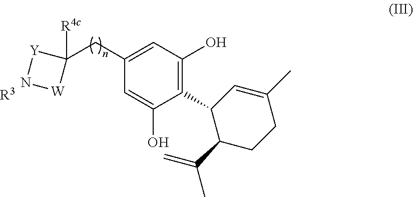

including hydrates, solvates, pharmaceutically acceptable salts, and complexes thereof, wherein R³, R^{4c}, Y, W, and n of formula (III) are as defined above with respect to formula (I). In certain embodiments of the invention, R³, R^{4c}, Y, W, and n of formula (III) are as defined in Table 2 below.

TABLE 2

| Entry | R³ | R^{4c} | Y | W | n |
|---|---|---|---|---|---|
| 1 | COCH₃ | H | CH₂ | CH₂ | 1 |
| 2 | CO₂CH₃ | H | CH₂ | CH₂ | 1 |
| 3 | CO₂ CH₂CH₃ | H | CH₂ | CH₂ | 1 |
| 4 | CON(CH₃)₂ | H | CH₂ | CH₂ | 1 |
| 5 | SO₂N(CH₃)₂ | H | CH₂ | CH₂ | 1 |
| 6 | SO₂ CH₃ | H | CH₂ | CH₂ | 1 |
| 7 | COCH₃ | H | CH₂ | CH₂CH₂ | 1 |
| 8 | CO₂CH₃ | H | CH₂ | CH₂CH₂ | 1 |
| 9 | CO₂ CH₂CH₃ | H | CH₂ | CH₂CH₂ | 1 |
| 10 | CON(CH₃)₂ | H | CH₂ | CH₂CH₂ | 1 |
| 11 | SO₂N(CH₃)₂ | H | CH₂ | CH₂CH₂ | 1 |
| 12 | SO₂ CH₃ | H | CH₂ | CH₂CH₂ | 1 |
| 13 | COCH₃ | H | CH₂CH₂ | CH₂ | 1 |
| 14 | CO₂CH₃ | H | CH₂CH₂ | CH₂ | 1 |
| 15 | CO₂ CH₂CH₃ | H | CH₂CH₂ | CH₂ | 1 |
| 16 | CON(CH₃)₂ | H | CH₂CH₂ | CH₂ | 1 |
| 17 | SO₂N(CH₃)₂ | H | CH₂CH₂ | CH₂ | 1 |
| 18 | SO₂ CH₃ | H | CH₂CH₂ | CH₂ | 1 |
| 19 | COCH₃ | H | CH₂CH₂ | CH₂CH₂ | 1 |
| 20 | CO₂CH₃ | H | CH₂CH₂ | CH₂CH₂ | 1 |
| 21 | CO₂ CH₂CH₃ | H | CH₂CH₂ | CH₂CH₂ | 1 |
| 22 | CON(CH₃)₂ | H | CH₂CH₂ | CH₂CH₂ | 1 |
| 23 | SO₂N(CH₃)₂ | H | CH₂CH₂ | CH₂CH₂ | 1 |
| 24 | SO₂ CH₃ | H | CH₂CH₂ | CH₂CH₂ | 1 |

The compounds of the invention further include enantiomers of compounds of the formula (III).

The compounds of the invention further include compounds of the formula (III) that are isotopically labeled with 1 to 10 deuterium atoms.

The compounds of the invention further include compounds having formula (IV):

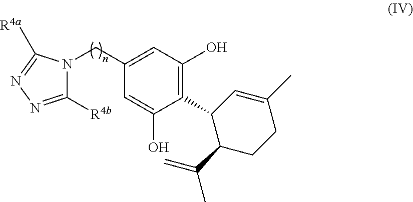

including hydrates, solvates, pharmaceutically acceptable salts, and complexes thereof, wherein n, R^{4a} and R^{4b} are as defined above with respect to formula (I).

The compounds of the invention further include enantiomers of compounds of the formula (IV).

The compounds of the invention further include compounds of the formula (IV) that are isotopically labeled with 1 to 10 deuterium atoms.

The compounds of the invention further include compounds having formula (V):

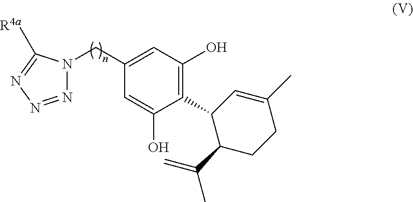

including hydrates, solvates, pharmaceutically acceptable salts, and complexes thereof, wherein n and R^{4a} are as defined above with respect to formula (I).

The compounds of the invention further include enantiomers of compounds of the formula (V).

The compounds of the invention further include compounds of the formula (V) that are isotopically labeled with 1 to 10 deuterium atoms.

The compounds of the invention further include compounds having formula (VI):

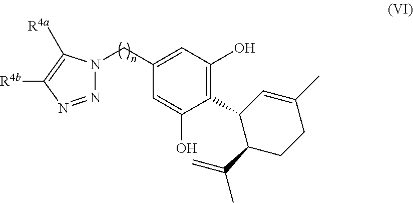

including hydrates, solvates, pharmaceutically acceptable salts, and complexes thereof, wherein n, R^{4a} and R^{4b} are as defined above with respect to formula (I).

The compounds of the invention further include compounds of the formula (VI) that are isotopically labeled with 1 to 10 deuterium atoms.

The compounds of the invention further include enantiomers of compounds of the formula (VI).

The compounds of the invention further include compounds having formula (VII):

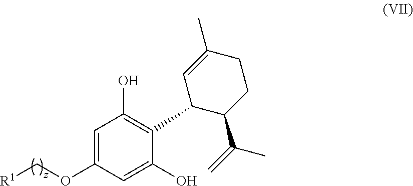

including hydrates, solvates, pharmaceutically acceptable salts, and complexes thereof, wherein R¹ and z are as defined above with respect to formula (I).

The compounds of the invention further include enantiomers of compounds of the formula (VII).

The compounds of the invention further include compounds of the formula (VII) that are isotopically labeled with 1 to 10 deuterium atoms.

The compounds of the invention further include compounds having formula (VIII):

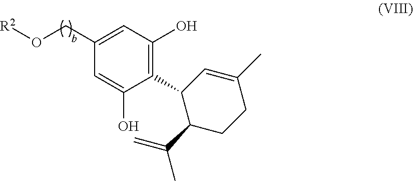

including hydrates, solvates, pharmaceutically acceptable salts, and complexes thereof, wherein R² and b are as defined above with respect to formula (I).

The compounds of the invention further include enantiomers of compounds of the formula (VIII).

The compounds of the invention further include compounds of the formula (VIII) that are isotopically labeled with 1 to 10 deuterium atoms.

The compounds of the invention further include compounds having formula (IX).

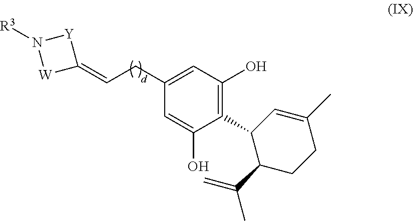

including hydrates, solvates, pharmaceutically acceptable salts, and complexes thereof, wherein R³, Y, W, and d of formula (IX) are as defined above with respect to formula (I). In certain embodiments of the invention, R³, Y, W, and d of formula (IX) are as defined in Table 3 below.

TABLE 3

| Entry | R³ | Y | W | d |
|---|---|---|---|---|
| 1 | COCH₃ | CH₂ | CH₂ | 1 |
| 2 | CO₂CH₃ | CH₂ | CH₂ | 1 |
| 3 | CO₂ CH₂CH₃ | CH₂ | CH₂ | 1 |
| 4 | CON(CH₃)₂ | CH₂ | CH₂ | 1 |
| 5 | SO₂N(CH₃)₂ | CH₂ | CH₂ | 1 |
| 6 | SO₂ CH₃ | CH₂ | CH₂ | 1 |
| 7 | COCH₃ | CH₂ | CH₂CH₂ | 1 |
| 8 | CO₂CH₃ | CH₂ | CH₂CH₂ | 1 |
| 9 | CO₂ CH₂CH₃ | CH₂ | CH₂CH₂ | 1 |
| 10 | CON(CH₃)₂ | CH₂ | CH₂CH₂ | 1 |
| 11 | SO₂N(CH₃)₂ | CH₂ | CH₂CH₂ | 1 |
| 12 | SO₂ CH₃ | CH₂ | CH₂CH₂ | 1 |
| 13 | COCH₃ | CH₂CH₂ | CH₂ | 1 |
| 14 | CO₂CH₃ | CH₂CH₂ | CH₂ | 1 |
| 15 | CO2 CH₂CH₃ | CH₂CH₂ | CH₂ | 1 |
| 16 | CON(CH₃)₂ | CH₂CH₂ | CH₂ | 1 |
| 17 | SO₂N(CH₃)₂ | CH₂CH₂ | CH₂ | 1 |
| 18 | SO₂ CH₃ | CH₂CH₂ | CH₂ | 1 |
| 19 | COCH₃ | CH₂CH₂ | CH₂CH₂ | 1 |
| 20 | CO₂CH₃ | CH₂CH₂ | CH₂CH₂ | 1 |
| 21 | CO₂ CH₂CH₃ | CH₂CH₂ | CH₂CH₂ | 1 |
| 22 | CON(CH₃)₂ | CH₂CH₂ | CH₂CH₂ | 1 |
| 23 | SO₂N(CH₃)₂ | CH₂CH₂ | CH₂CH₂ | 1 |
| 24 | SO₂ CH₃ | CH₂CH₂ | CH₂CH₂ | 1 |

The compounds of the invention further include enantiomers of compounds of the formula (IX).

The compounds of the invention further include compounds of the formula (IX) that are isotopically labeled with 1 to 10 deuterium atoms.

The compounds of the invention further include compounds having formula (X):

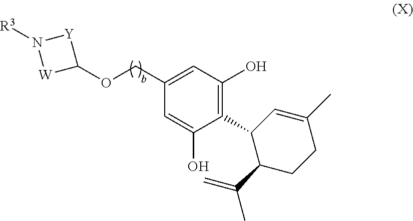

including hydrates, solvates, pharmaceutically acceptable salts, and complexes thereof, wherein R³, Y, W, and b of formula (X) are as defined above with respect to formula (I). In certain embodiments of the invention, R³, Y, W, and b of formula (X) are as defined in Table 4 below.

TABLE 4

| Entry | R³ | Y | W | b |
|---|---|---|---|---|
| 1 | COCH₃ | CH₂ | CH₂ | 1 |
| 2 | CO₂CH₃ | CH₂ | CH₂ | 1 |
| 3 | CO₂ CH₂CH₃ | CH₂ | CH₂ | 1 |
| 4 | CON(CH₃)₂ | CH₂ | CH₂ | 1 |
| 5 | SO₂N(CH₃)₂ | CH₂ | CH₂ | 1 |
| 6 | SO₂ CH₃ | CH₂ | CH₂ | 1 |
| 7 | COCH₃ | CH₂ | CH₂CH₂ | 1 |
| 8 | CO₂CH₃ | CH₂ | CH₂CH₂ | 1 |
| 9 | CO₂ CH₂CH₃ | CH₂ | CH₂CH₂ | 1 |
| 10 | CON(CH₃)₂ | CH₂ | CH₂CH₂ | 1 |
| 11 | SO₂N(CH₃)₂ | CH₂ | CH₂CH₂ | 1 |
| 12 | SO₂ CH₃ | CH₂ | CH₂CH₂ | 1 |
| 13 | COCH₃ | CH₂CH₂ | CH₂ | 1 |
| 14 | CO₂CH₃ | CH₂CH₂ | CH₂ | 1 |
| 15 | CO₂ CH₂CH₃ | CH₂CH₂ | CH₂ | 1 |
| 16 | CON(CH₃)₂ | CH₂CH₂ | CH₂ | 1 |
| 17 | SO₂N(CH₃)₂ | CH₂CH₂ | CH₂ | 1 |
| 18 | SO₂ CH₃ | CH₂CH₂ | CH₂ | 1 |
| 19 | COCH₃ | CH₂CH₂ | CH₂CH₂ | 1 |
| 20 | CO₂CH₃ | CH₂CH₂ | CH₂CH₂ | 1 |
| 21 | CO₂ CH₂CH₃ | CH₂CH₂ | CH₂CH₂ | 1 |
| 22 | CON(CH₃)₂ | CH₂CH₂ | CH₂CH₂ | 1 |
| 23 | SO₂N(CH₃)₂ | CH₂CH₂ | CH₂CH₂ | 1 |
| 24 | SO₂ CH₃ | CH₂CH₂ | CH₂CH₂ | 1 |

The compounds of the invention further include enantiomers of compounds of the formula (X).

The compounds of the invention further include compounds of the formula (X) that are isotopically labeled with 1 to 10 deuterium atoms.

For the purposes of demonstrating the manner in which the compounds of the invention are named and referred to herein, the compound having the formula:

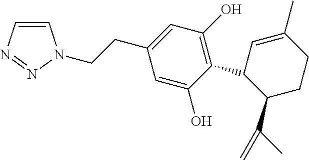

has the chemical name 5-(2-(1H-1,2,3-triazol-1-yl)ethyl)-2-((1R,6R)-3-methyl-6-(prop-1-en-2-yl)cyclohex-2-enyl)benzene-1,3-diol, and is sometimes referred to herein as KLS-13007.

For the purposes of demonstrating the manner in which the compounds of the invention are named and referred to herein, the compound having the formula:

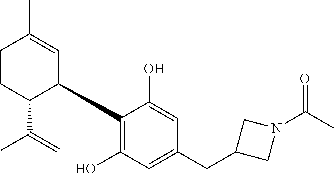 [SMILES: C=C(C)[C@@H]1CCC(C)=C[C@H]1c1c(O)cc(CC2CN(C(C)=O)C2)cc1O]

has the chemical name 1-(3-(3,5-dihydroxy-4-((1R,6R)-3-methyl-6-(prop-1-en-2-yl)cyclohex-2-enyl)benzyl)azetidin-1-yl)ethenone, and is sometimes referred to herein as KLS-13019.

For the purposes of demonstrating the manner in which the compounds of the invention are named and referred to herein, the compound having the formula:

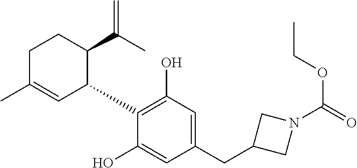 [SMILES: C=C(C)[C@@H]1CCC(C)=C[C@H]1c1c(O)cc(CC2CN(C(=O)OCC)C2)cc1O]

has the chemical name ethyl 3-(3,5-dihydroxy-4-((1R,6R)-3-methyl-6-(prop-1-en-2-yl)cyclohex-2-enyl)benzyl)azetidine-1-carboxylate, and is sometimes referred to herein as KLS-13022.

Compositions of the Invention

The present invention also relates to compositions or formulations which comprise the functionalized 1,3-benzenediols according to the invention. In general, the compositions of the invention comprise an effective amount of at least one functionalized 1,3-benzenediol and/or a salt thereof and at least one excipient.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the invention have improved cellular potency, pharmacokinetic properties, as well as improved oral bioavailability.

Examples of suitable excipients are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes.

As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the invention can be administered topically or parenterally, neat or in combination with conventional pharmaceutical carriers. Applicable solid carriers can include one or more substances which can also act as lubricants, solubilizers, suspending agents, fillers, glidants, compression aids, binders or disintegrating agents, or encapsulating materials. The compounds can be formulated in conventional manner, for example, in a manner similar to that used for known therapeutic agents. In powders, the carrier can be a finely divided solid, which is an admixture with a finely divided compound.

Liquid carriers can be used in preparing solutions, suspensions and emulsions. A compound of the invention can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, suspending agents, thickening agents, colors, viscosity regulators, stabilizers, and osmo-regulators. Examples of liquid carriers for topical and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection.

In certain embodiments, the pharmaceutical composition is in unit dosage form. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Such unit dosage form can contain from about 1 mg/kg of compound to about 500 mg/kg of compound, and can be given in a single dose or in two or more doses. Such doses can be administered in any manner useful in directing the compound(s) to the recipient's skin or ocular tissue, including topically or parenterally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the invention can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

Compounds described herein can be administered parenterally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions and solutions.

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable.

To increase the effectiveness of compounds of the invention, it can be desirable to combine a compound with other agents effective in the treatment of the target disorder. For example, other active compounds (i.e., other active ingredients or agents) effective in treating the target disorder can be administered with compounds of the invention. The other agents can be administered at the same time or at different times than the compounds disclosed herein.

Compounds and compositions of the invention can be useful for the treatment or inhibition of a pathological condition or disorder in a mammal, for example, a human subject.

Non-limiting examples of compositions according to the invention include from about 0.001 mg to about 1000 mg of one or more functionalized 1,3-benzene diols according to the present invention and one or more excipients; from about 0.01 mg to about 100 mg of one or more functionalized 1,3-benzenediols according to the invention and one or more excipients; and from about 0.1 mg to about 10 mg of one or more functionalized 1,3-benzenediols according to the invention; and one or more excipients.

### Method of the Invention

Pathogenesis and inflammation of RT: The pathogenesis of radiation dermatitis is not fully understood, although it is clear that excessive release of reactive oxygen species (ROS), proteases and inflammatory mediators damage the surrounding tissues after RT. It has been proposed that TRPM2 (transient receptor potential melastatin-2) plays a significant role in RD and that this TRP channel may mediate a portion of the inflammatory response (Perraud et al., 2019). TRPM2 is activated under oxidative stress and stimulates hydrogen peroxide-induced calcium influx which also may play a role in radiation dermatitis. In addition, a number of known cell signaling pathways have been associated with radiation dermatitis that include: the nuclear factor κβ pathway and mitogen-activated protein kinase pathway. Activation of these pathways leads to the release of proinflammatory cytokines that include IL-1β, IL-6 and TNFα. Importantly, the release of all three of these inflammatory cytokines are inhibited from various skin cell cultures after treatment with KLS-13022 as shown in this disclosure.

In this disclosure, KLS-13022 is shown to decrease the release of IL-8 from human dermal microvascular endothelial cells after co-treatment with nickel sulfate. These studies on nickel-induced allergy strongly indicate that the potential therapeutic effects of the 1,3-benzene diols extend beyond radiation dermatitis to include hypersensitivity to metals.

The previously disclosed 1,3-benzene diols (Kinney et al., 2016) and their protection of nerve cells are now extended to dermatological remedies that include but are not limited to: photoaging, melasma, non-melanoma skin cancer, psoriasis, alopecia, vitiligo, systemic sclerosis, actin keratosis, radiation dermatitis and atopic dermatitis.

The invention comprises a method for treating or preventing dermatological disorders with functionalized 1,3-benzene diol compounds of the invention.

In certain embodiments, the method comprises treating or preventing pain and oxidative stress in skin tissue after irradiation of a subject in need thereof.

In certain embodiments, the method treats or prevents pain and symptoms (rashes, redness or bumps) in skin tissue after exposure to metals producing allergies in a subject.

In certain embodiments, the method further includes the administration of functionalized 1,3-benzene diol compounds in effective concentrations for treating irradiated skin tissue that do not produce toxicity of epidermal keratinocytes, dermal Microvascular endothelial cells or dermal fibroblast at concentrations that are less than 20 times the effective therapeutic dose.

In certain embodiments, the functionalized 1,3-benzene diol compounds exhibit antioxidant activity that is effective in treating irradiated skin tissue.

In certain embodiments, the functionalized 1,3-benzene diol compounds exhibit therapeutic effects by locally restricted tissue actions that do not involve significant entry in the systemic circulation.

In certain embodiments, the functionalized 1,3-benzene diol compounds are therapeutically effective for irradiated skin and are provided in a formulation that both solubilizes and enhances protection against oxidative damage.

In certain embodiments, the method comprises the co-administration of functionalized 1,3-benzene diol compounds and soy lecithin. The soy lecithin preferably comprises phosphatidylcholine with a polyunsaturation of the fatty acyl composition of at least 63%.

In certain embodiments, the method of the invention comprises treating or preventing oxidative stress and allergy in skin tissues.

The invention will be illustrated in more detail with reference to the following Examples, but it should be understood that the invention is not deemed to be limited thereto.

### EXAMPLES

Procedures

The following procedures can be utilized in evaluating and selecting compounds as protective agents against radiation and oxidative stress for dermatological treatments.

Cell Cultures were utilized to establish the protective properties of 1,3-benzene diols in skin models relevant to human treatments and to determine concentrations that were non-toxic.

Normal human epidermal keratinocytes cultures were utilized to measure the release of proinflammatory cytokines and chemokines. Primary epidermal keratinocytes from neonatal donors were used from human origin and purchased from Thermo-Fisher (Cat No. A13401). Cell cultures were initiated following manufacturer suggested protocols using HKGS-supplemented (Cat No. S0015) EPILIFE medium (Cat No. MEPI500CA). Cells were cultured until the second passage and then acclimated for 48 hours more before UV-B irradiation. Dosing procedures and cell groups were identified using appropriate cell culture 24-well plates. The cell culture incubator was set at 5% v/v CO₂ with temperature and humidity maintained at 36-38° C. and 40-70%, respectively. The test and control substances were administered to HKGS-depleted medium in triplicates. A fixed 5 μL/well from 100× stock dose volume was administered to 500 μL of media to each cell culture well on Day 3 and pre-incubated for 6 hours and then post-UVB incubated for 18 hours. The same procedure was utilized to determine non-toxic concentrations of 1,3-benzene diols after 24 hours of treatment.

Dermal Microvascular endothelial cells of human origin were cultured in vitro using standard conditions (37° C., 95% v/v air, 5% v/v CO₂). Human cells were selected as relevant to cell types within skin tissues for this development program. Cell culture methods are commonly used in non-clinical studies conducted on pharmaceutical products. Cell cultures were initiated following manufacturer suggested protocols using growth factor-supplemented EGM-2 Endothelial Cell Growth Medium-2 (Lonza, Morristown, N.J.). Cells were cultured until the third and fourth passage and then acclimated for 24 hours before Nickel induction and dosing procedures. Cell treatment groups were identified using appropriate 96-well plates. The test and control articles were administered to medium containing 1 mM NiSO₄ in triplicates. The negative control group received vehicle-only (0.1% v/v final) dose. A fixed 0.1 μL/well from 1000× stock dose volume was administered to 100 μL of media to each cell culture well on Day 2 and incubated for 6 hours at 37 degrees. These cell cultures were also utilized to determine the concentrations of test agent that were non-toxic after 24 hours of incubation.

Primary dermal fibroblasts cells of human origin were cultured in vitro using standard conditions (37° C., 95% v/v air, 5% v/v CO₂). Primary dermal fibroblasts from neonatal donor were used from human origin and purchased from Thermo-Fisher (Cat No. C0045C). Cell cultures were initiated following manufacturer suggested protocols using Fetal Bovine Serum-supplemented (Cat No. A3840202) DMEM medium (Cat No. 11995065). Cells were cultured until the second passage and then acclimated for 48 hours before UVA irradiation and dosing procedures conducted in 24-well plates. On Day 3, test materials were dosed to cell culture media and pre-incubated for 6 hours. Treatment media was then removed and replaced with phosphate buffer saline (PBS) without test materials and Ultraviolet-A (UV-A) was irradiated at 12.5 J/cm² using FS-20/T-12 bulbs (315-400 nm). After UV-A irradiation, PBS was replaced with fresh DMEM medium with test materials and incubated for 18 hours at standard culture conditions. After incubation, culture medium supernatants were harvested and used to measure human IL-6 levels by ELISA method. In vitro dosing route was selected to test the activity of KLS-13022 after pro-inflammatory inducer UV-A irradiation, since isolated human cell culture provide direct activity indication and specific cell-type effects. Human cells were selected as relevant to cell types within skin tissues for this development program. Cell culture methods are commonly used in nonclinical studies. A fixed 0.5 μL/well from 1000× stock dose volume was administered to 500 μL of media to each cell culture well on Day 3 and pre-incubated for 6 hours at 37° C. and 5% CO2 and later post-UVA incubated for 18 hours.

Assays of proinflammatory cytokines TNFα and IL10: Test materials were applied in vitro for the ability to inhibit pro-inflammatory (TNFα and IL-1) cytokine secretion by cultured NHEKs irradiated with UV-B. On Day 1, primary NHEKs were seeded in 24-well plates and cultured for 48 hours before treatments. On Day 3, cells were pre-treated with test and control material, treatment media was removed and cells were irradiated with Ultraviolet-B (UVB) at 25 mJ/cm² (280-312 nm). After irradiation, media with test and control material were added and cells were incubated for an additional 18 hours. Media supernatants were harvested and analyzed for TNFα and IL-1β levels by ELISA method. Cytokine levels were assessed from cell culture media supernatants using BD Biosciences OptEIA™ Human TNFα (Cat. No. 555212) and IL-1β (Cat. No. 557953) and ELISA set kit and Reagent Set B (Cat. No. 550534) according the manufacture's specifications. Absorbance was measured at 450 nm using a plate reader and calculated cytokine levels were determined from a standard curve.

Assay of CXCL5: Test materials were applied in vitro for the ability to inhibit CXCL5 chemokine secretion as peripheral mediator of UV-B-induced inflammatory pain by cultured NHEKs. Piroxicam was tested as control material for analgesic activity. CXCL5 levels were quantified by enzyme-linked immunosorbent assay (ELISA) after UV-B irradiation and incubation with test and control materials. CXCL5 levels were assessed from cell culture media supernatants using R&D Systems Human CXCL5/ENA-78 DuoSet ELISA kit (Cat. No. DY254-05) according to the manufacture's specifications. Absorbance was measured at 450 nm using plate reader and cytokine levels were calculated from a standard curve.

Assay for Interleukin-8: Test material was applied in vitro for the ability to inhibit pro-inflammatory (IL-8) cytokine secretion by cultured HDMECs co-treated with Nickel (NiSO4). Clobetasol propionate was tested as positive control material for anti-inflammatory activity. The pro-inflammatory cytokine (IL-8) was quantified by enzyme-linked immunosorbent assay (ELISA) after inflammatory induction and incubation with test and control materials. Interleukin-8 levels in cell culture medium are markers of inflammation signaling by endothelial cells present in skin microvascular tissue in response to irritation. Interleukin-8 (IL-8) levels were assessed using BD Biosciences OptEIA™ Human IL-8 ELISA Set kit (Cat. No. 555244) and Reagent Set B (Cat. No. 550534) according to the manufacture's specifications. Absorbance was measured at 450 nm using plate reader and interleukin-8 levels were calculated from a standard curve.

An Interleukin-6 assay was used to evaluate the anti-inflammatory activity of KLS-13022 formulated in ethanol and applied in vitro to cells irradiated with Ultraviolet-A (UV-A)-induced photoaging (Wlascheck et al., 1994) in human dermal fibroblasts (HDFs). Test materials were applied in vitro for the ability to inhibit pro-inflammatory (IL-6) cytokine secretion by cultured HDFs irradiated with UV-A. Alpha-Tocopherol (vitamin E) was tested as control material for anti-photoaging activity. IL-6 levels were quantified by enzyme-linked immunosorbent assay (ELISA) after inflammatory induction and incubation with test and control materials. Interleukin-6 (IL-6) levels were assessed from cell culture media supernatants using BD Biosciences OptEIA™ Human IL-6 ELISA Set kit (Cat. No. 555220). according to the manufacture's specifications. Absorbance was measured at 450 nm by a plate reader and interleukin-6 levels were calculated from a standard curve.

Cell Viability assays were conducted using MTS reduction assays (Promega) diluted 1:5 in DMEM+10% FBS media as specified by the manufacturer. Absorbance was measured at 490 nm using plate reader and cell viability was calculated as a percent of control. This assay was done along with all culture systems that were utilized to study cytokine release. Similarly, the MTT reduction assay was conducted as specified from the manufacturer (Sigma Aldrich).

Cell Irritancy with EPI-DERM protocol. The purpose of this study was to evaluate the potential skin irritation activity of KLS-13022 formulations compared to vehicle-only treated tissues in vitro on human 3D skin-like EPIDERM by topical application according to OECD 439 protocol. Primary skin-like inserts were used from human origin and purchased from MatTek (Ashland, Mass.). Tissue cultures were initiated following manufacturer suggested protocols using provided growth medium from kit (Cat No. EPI-100-NMM-SIT). Test formulations of KLS-13022 up to 3% w/v in 60:40 ethanol:water vehicle were prepared fresh before use. EPIDERM tissues were acclimated before treatments for 24 hours and later treated topically for 1 hour with KLS-13022 (0.01, 0.1, 0.3 1, 3% w/v). Sodium dodecyl sulfate (SDS) was used as positive skin irritant control. Tissue viability levels were measured by MTT reduction assay method after material treatments. The levels of tissue viability after each material treatment was compared to vehicle control group to determine any potential skin irritation activity. Average levels of tissue viability were calculated for each treatment group. According to OECD 439 protocol, predicted positive skin irritation activities were determined when obtaining <50% tissue viability. Results produced by MTT reduction assay showed that treatments with SDS produced a significant decrease in tissue viability; therefore this substance is a strong skin irritant. Conversely, KLS-13022 up to 3% w/v in 60:40 ethanol:water formulation did not produced irritancy potential based on ≥50% tissue viability and not significantly changed tissue viability compared to vehicle-only treated tissues. Tissues were cultured in standard conditions (37° C., 95% v/v air, 5% v/v CO2) and acclimated for 18+3 hours before dosing procedures.

Ocular irritancy: The purpose of this study was to evaluate any potential ocular irritation activity of KLS-13022 formulations compared to vehicle-only treated tissues in vitro on human 3D cornea-like EPIOCULAR by topical application according to OECD 492 protocol. Test formulations of KLS-13022 up to 3% w/v in 60:40 ethanol:water vehicle were prepared fresh before use. EPIOCULAR tissues were acclimated before treatments for 24 hours and later treated topically for 30 minutes with KLS-13022 (0.01, 0.1, 0.3 1, 3% w/v). Methyl acetate was used as positive eye irritant control. Tissue viability levels were measured by MTT reduction assay method after material treatments. The levels of tissue viability after each material treatment was compared to vehicle control group to determine any potential ocular irritation activity.

Average levels of tissue viability were calculated for each treatment group. According to OECD 492 protocol, predicted positive eye irritation activities were determined when obtaining <60% tissue viability. Results produced by MTT reduction assay showed that treatments with methyl acetate produced a significant decrease in tissue viability; therefore this substance is a strong eye irritant. Conversely, KLS-13022 up to 3% w/v in 60:40 ethanol:water formulation did not produced irritancy potential based on ≥60% tissue viability and not significantly changed tissue viability compared to vehicle-only treated tissues. Primary cornea-like inserts were used from human origin and purchased from MatTek (Ashland, Mass.). Tissue cultures were initiated following manufacturer suggested protocols using provided growth medium form kit (Cat No. OCL-200-EIT). Tissues were cultured with standard conditions (37° C., 95% v/v air, 5% v/v CO2) and acclimated for 18+3 hours before dosing procedures.

Antioxidant capacity: The purpose of this study was to evaluate the overall antioxidant capacity of KLS-13022 for the ability to inhibit the oxidation of ABTS. (2,2′-Azino-di-[3-ethylbenzthiazoline sulphonate]) to ABTS.⁺ by metmyoglobin. Ascorbic acid (Vitamin C) was used as positive antioxidant control in the assay. The testing involved the suppression of optical density (OD) at 750 nm to a degree, which is proportional to an effective oxidation inhibition. Neat solutions were prepared for materials formulated in HPLC-grade water (ascorbic acid) or 200-proof ethanol (KLS-13022). Serial dilutions were prepared for each material and mixed with metmyoglobin enzyme in assay buffer solution. All samples were compared to vehicle-only treated samples used as negative oxidation inhibition. Optical density changes at 750 nm were evaluated 15 minutes after addition of hydrogen peroxide to each sample. Average absorbance was calculated and plotted for each sample concentration. Effective antioxidant inhibition activity based on the change of absorbance of blank (vehicle-only) sample (no material added). Results produced by optical density changes for each test material showed that KLS-13022 provided strong antioxidant activity with an average EC₅₀ effective activity of 25.6 μM compared to ascorbic acid (EC₅₀=39.7 μM). The Antioxidant assay kit was obtained from Cayman Chemical Co. (Ann Arbor, Mich.). The assay consisted in the evaluation of the overall antioxidant capacity of test materials for the ability to inhibit the oxidation of ABTS. to ABTS.⁺ by metmyoglobin. The testing involved the suppression of optical density (OD) at 750 nm to a degree which is proportional to an effective oxidation inhibition. The antioxidant capacity of each material was compared to ascorbic acid (vitamin C) used as standard control. Test material solutions were prepared for each material formulated in ethanol or HPLC-grade water. Seven (7) two-fold serial dilutions were made from the stock solution samples. Negative control wells received vehicle-only.

Percutaneous absorption (Franz cell). For these studies, porcine ear skin (n=3; 1.5 in²) obtained from Animal Technologies, Inc. (Tyler, T X) were utilized, Skins were thawed for 30 min at room temperature. After thawing, skin was placed over receptor compartment of the unjacketed Franz cell chamber with stir bar obtained from Permegear and covered with the donor compartment. The clamp was then placed to hold the cell together. The receptor compartment was filled with 12 mL of 1M PBS buffer+4% BSA and 0.1% sodium azide through the sampling port using a syringe. Franz cell was placed on a stir plate at ˜600 RPM and heated till the buffer reached 37° C. 400 μL of 3H—H₂O (˜1.5 million counts) was then placed on the membrane for 5 min. Following, the 3H—H₂O was removed and the membrane was washed with water. After 30 min, 250 μL of the receptor buffer was removed in triplicate and counted to check the integrity of the skin (>0.4 μl/cm² was considered acceptable). The volume removed from the receptor compartment was replaced with fresh buffer once the skin integrity was validated. For testing, 7.5 mg/cm² of the KLS-13022 formulation was then placed on the membrane and receptor buffer removed in its entirety after 24 hours. At the end of the assay, wash was performed using 2×0.5 ml of 50% ethanol and pipetting up and down 10 times. After the wash, 20 tape strips (using 3M TRANSPORE tape) were performed to remove stratum corneum. The remaining skin tissue was cut so that only the circular portion the formulation remained. Extraction and homogenization were performed to quantify the amount of formulation remaining in the epidermis and dermis. After removal of stratum corneum by tape strips, the circular portion of skin tissue was cut into fine pieces with scissors and transferred into a glass tube containing 2.0 ml of 50% ethanol. The cut skin was homogenized. The tubes were mixed for 4 hours at room temperature and then centrifuged for 10 min at 14000 rpm. The supernatant was transferred into a clean vial and 10 μl skin extract was used for HPLC analysis. The concentration of KLS-13022 was measured by liquid chromatography coupled with tandem mass spectrometry (LC/MS/MS). Verapamil was used as internal standard. The study samples were diluted with the mixture of ethanol and water (50:50), followed by submission to Kromasil C18 HPLC column. The analytes and internal standard were detected using an Applied Biosystem API-3000 mass spectrometer equipped with a Turbo-Ion Spray (Electrospray, ESI) source that was operated in multiple reaction monitoring mode (MRM) under positive ion mode.

Results

FIG. 1 shows the decreased release of TNFα into a culture medium of human epidermal keratinocytes irradiated with ultraviolet-B after treatment with various concentrations of KLS-13022. In particular, FIG. 1 shows TNFα levels secreted into the medium after Ultraviolet-B (UV-B) irradiation (25 mJ/cm² at 280-312 nm)) and KLS-13022 treatment of human epidermal keratinocytes. Data represent mean±SEM from 3 independent experiments. Statistical comparisons were made to UV-B+control group (*p<0.01). Based on these data, it was concluded that KLS-13022 provided a positive anti-inflammatory activity based on TNFα inhibition against UV-B irradiation in cultured human epidermal keratinocytes. The IC50 of this effect was determined to be 0.38 μM. The upper reference line of dot-dash represents the value of cultures treated with UV-B in the ethanol vehicle. The final ethanol vehicle did not exceed 0.3% for any of the KLS-13022 concentrations tested.

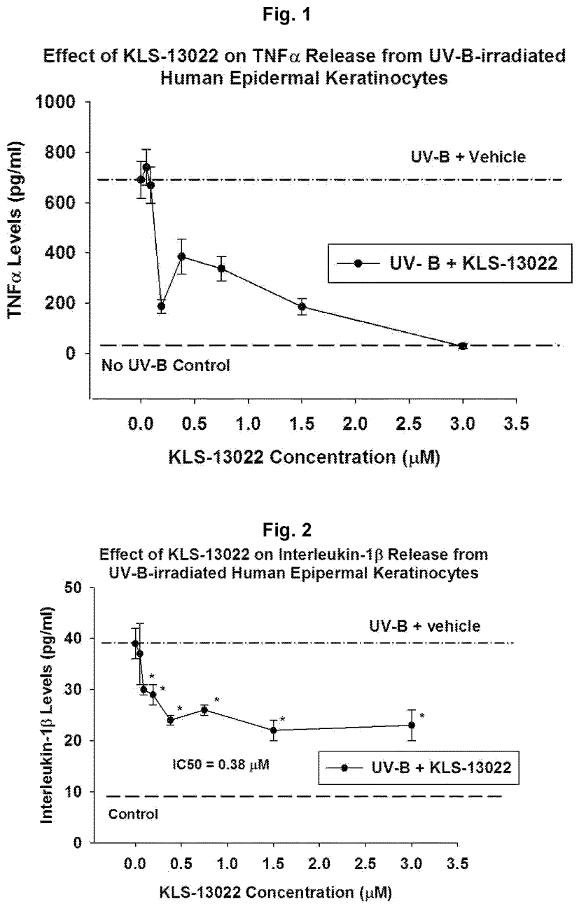

FIG. 2 shows the decreased release of Interleukin-1p into the culture medium of human epidermal keratinocytes irradiated with ultraviolet-B after treatment with various concentration of KLS-13022. Interleukin-1l levels are shown in cultures after UV-B irradiation (25 mJ/cm²) and KLS-13022 treatment in human epidermal keratinocytes. Data represent mean±SEM from 3 independent experiments. Statistical comparisons were made to UV-B+vehicle control group (* p<0.01). Based on these data, it was concluded that KLS-13022 provided a positive anti-inflammatory activity based on interleukin-β inhibition against UV-B irradiation in cultured human epidermal keratinocytes. The IC50 of this effect was determined to be 0.38 μM. The upper reference line of dot-dash represents the value of cultures treated with UV-B in the ethanol vehicle. The lower reference line represents the value of cultures from untreated control cultures. The final ethanol vehicle did not exceed 0.3% for any of the KLS-13022 concentrations tested.

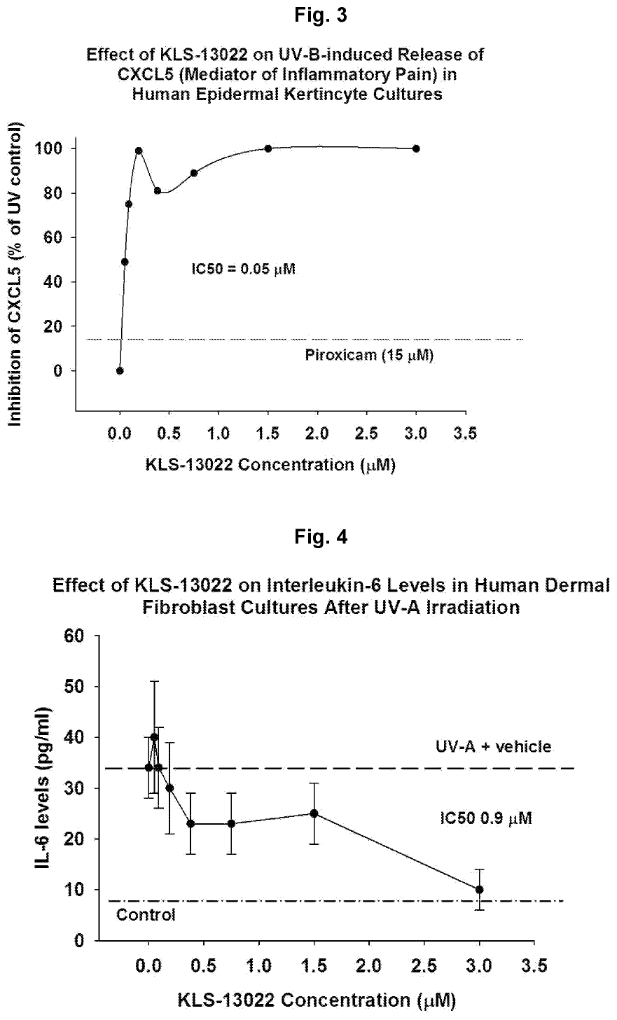

FIG. 3 shows the decreased release of the chemokine CXCL5 into the culture medium of normal human epidermal keratinocytes (NHEKs) irradiated with ultraviolet-B after co-treatment with various concentration of KLS-13022. The test material was applied for the ability to inhibit CXCL5 chemokine secretion as peripheral mediator of UVB-induced inflammatory pain by cultured NHEKs. CXCL5 levels were calculated after UV-B irradiation plus treatments to calculate effective analgesic activities. Inhibition data was evaluated by comparing the levels of CXCL5 in UV-B-irradiated and non-irradiated samples. Chemokine levels changes for UV-B plus Piroxicam were used as positive control. KLS-13022 provided 100% CXCL5 inhibition with IC₅₀=0.05 μM. Based on these data, it was concluded that KLS-13022 provided a positive analgesic activity based on CXCL5 inhibition against UVB-induced inflammatory pain in cultured human epidermal keratinocytes. The dashed reference line represents the values of from the piroxicam-treated positive control cultures with UV-B radiation.

FIG. 4 shows the decreased release of interleukin-6 into the culture medium of ultraviolet-A-induced photoaging in human dermal fibroblast cells after treatment with KLS-13022. IL-6 levels were calculated after UVA irradiation measure effective anti-inflammatory activities. Human dermal fibroblasts were irradiated with Ultraviolet-A (UV-A) at 12.5 J/cm² (315-400 nm). Inhibition data was evaluated by comparing the levels of IL-6 in UV-A-irradiated and non-irradiated samples. KLS-13022 provided a maximum 95% IL-6 inhibition with IC₅₀=0.9 μM. Based on these data, it was concluded that KLS-13022 provided a positive photoaging with anti-inflammatory activity based on IL-6 inhibition against UV-A irradiation in cultured human dermal fibroblasts cells. The upper dashed reference line represents the value of cultures treated with UV-A in the ethanol vehicle. The lower dot-dashed line represents the value of untreated control cultures. Data represent mean±SEM from 3 independent experiments. Significance compared to UVA+ethanol control group (* p<0.01).

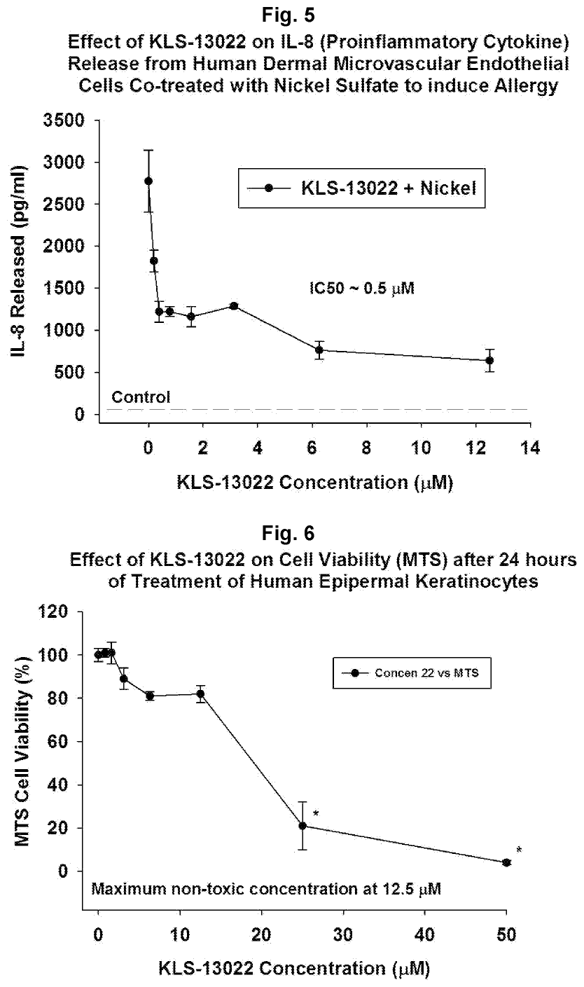

FIG. 5 shows the decreased release of interleukin-8 into the culture medium of nickel-induced allergy in human dermal microvascular endothelial cells after co-treatment with various concentrations of KLS-13022. The purpose of this study was to evaluate the anti-inflammatory activity of KLS-13022 applied in vitro to human dermal microvascular endothelial cells using Nickel (Ni²⁺)-induced allergy as Toll-like Receptor-4 (TLR-4) receptor inducer (Schmidt et al., 2010). IL-8 levels were calculated after NiSO4 to calculate effective anti-inflammatory activities. Inhibition data was evaluated by comparing the levels of IL-8 in vehicle-only treated and NiSO4+vehicle-only samples. Cytokine levels changes for nickel plus Clobetasol propionate were used as positive control showed that this compound produced anti-inflammatory effect with IC₅₀˜0.5 μM. KLS-13022 provided a maximum 78% IL-8 inhibition with IC₅₀=0.3 μM, suggesting a similar anti-inflammatory activity. Results represent Avg±SEM cumulative data from 3 independent experiments run in triplicates. IL-8 levels were determined by ELISA. Based on these data, it was concluded that KLS-13022 provided a positive anti-Nickel allergy with anti-inflammatory activity based on IL-8 inhibition against Nickel exposure in cultured human dermal microvascular endothelial cells.

FIG. 6 shows cell viability of human epidermal keratinocytes after treatment with various concentrations of KLS-13022. As measure of safety, the effect of KLS-13022 alone was measured with a MTS cell viability dye. Average levels of cell viability after KLS-13022 treatments were calculated based on vehicle-only treated group. Results shows that KLS-13022 exhibited non-toxic concentration ranges after 24 hours at ≤10 μM. * Non-toxic concentration determined by ≥80% viability after 24 hours treatments measured by MTS reduction assay. Results represent average from triplicate determinations.

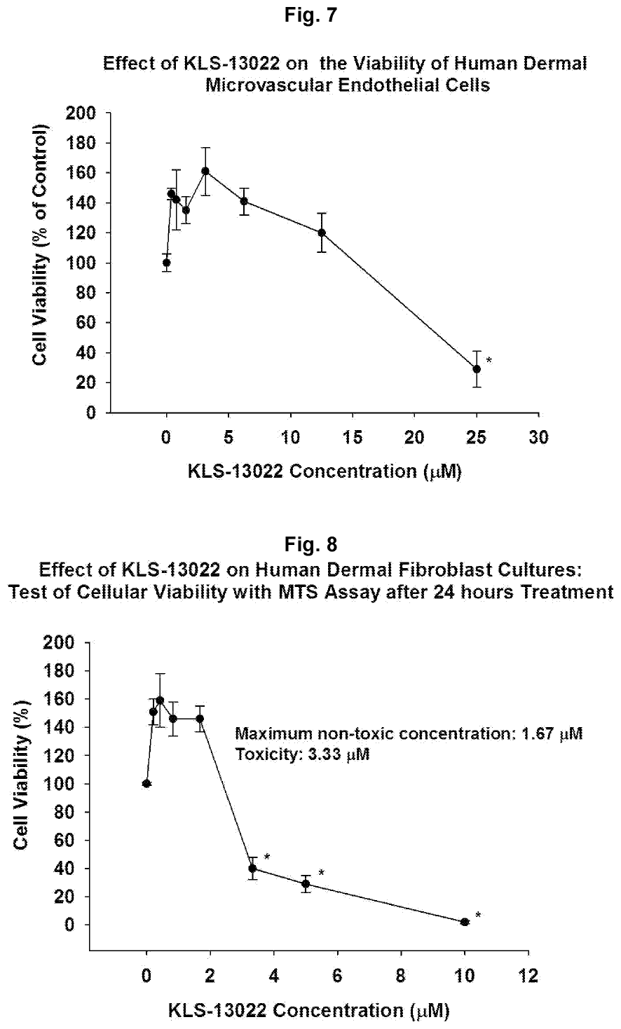

FIG. 7 show cell viability of human dermal microvascular endothelial cells after treatment with various concentrations of KLS-13022. As measure of safety, the effect of KLS-13022 alone was measured with a MTS cell viability dye. Average levels of cell viability after KLS-13022 treatment were calculated based on comparison to the vehicle-only treated group. Results show that KLS-13022 produced non-toxic concentration ranges ≤ to 12 μM after 6 hours incubation. * Toxic concentration was determined to be ≤20% viability after 6 hours treatments as measured by MTS reduction assay. Results represent average from triplicate determinations.

FIG. 8 shows cell viability of human dermal fibroblast cells after treatment with various concentrations of KLS-13022. As a measure of safety, the effect of KLS-13022 alone was measured with an MTS cell viability dye. Average levels of cell viability after KLS-13022 treatment were calculated based on comparison to the vehicle-only treated group. Results show that KLS-13022 produced non-toxic concentration ranges ≤1.67 μM after 24 hours incubation. KLS-13022 was toxic at 3.3 μM. * Toxic concentrations were determined by <40% viability after 24 hours treatments as measured by MTS reduction assay. Results represent average from triplicate determinations.

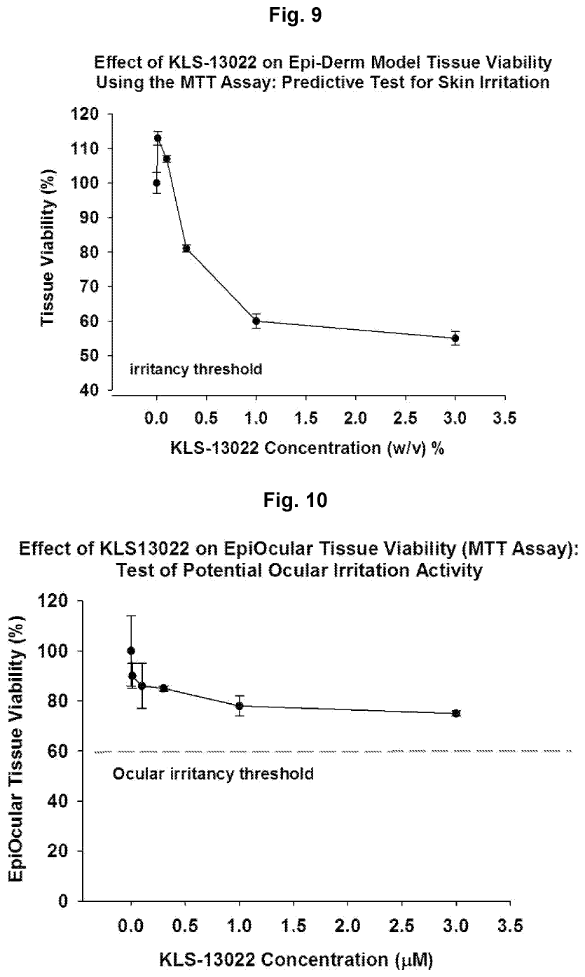

FIG. 9 shows the effect of KLS-13022 on potential skin irritation activity as determined with the human skin-like EPIDERM in vitro model. Potential skin irritation activity of KLS-13022 was evaluated by comparison to vehicle-only treated tissues in vitro on human 3D skin-like EPIDERM. According to OECD (Organization for Economic Cooperation and Development) 439 protocol, predicted positive skin irritation activities were determined when observing <50% tissue viability. Results produced by MTT reduction assay showed that treatments with SDS produced a significant decrease in tissue viability, therefore this substance is a strong skin irritant (data now shown). Conversely, KLS-13022 up to 3% w/v in 60:40 ethanol:water formulation did not produce irritancy potential based on ≥50% tissue viability and not significantly changed tissue viability compared to vehicle-only treated tissues.

FIG. 10 shows the effect of KLS-13022 on the in vitro eye irritation activity as determined in the reconstructed human cornea-like model-EPIOCULAR. The purpose of this study was to evaluate any potential ocular irritation activity of KLS-13022 formulations compared to vehicle-only treated tissues in vitro on human 3D cornea-like EPIOCULAR by topical application according to OECD 492 protocol. Test formulations of KLS-13022 up to 3% w/v in 60:40 ethanol:water vehicle. Methyl acetate was used as positive eye irritant control. Tissue viability levels were measured by MTT reduction assay, The levels of tissue viability after each material treatment was compared to vehicle control group to determine any potential ocular irritation activity.

Average levels of tissue viability were calculated. According to OECD 492 protocol, predicted positive eye irritation activities were determined when obtaining <60% tissue viability. Results produced by MTT reduction assay showed that treatments with methyl acetate produced a significant decrease in tissue viability, therefore this substance is a strong eye irritant (data not shown). KLS-13022 up to 3% w/v in 60:40 ethanol:water formulation did not produced irritancy potential based on ≥60% tissue viability and not significantly changed tissue viability compared to vehicle-only treated tissues. Based on these data, it was concluded that KLS-13022 formulated in 60:40 ethanol:water formulation is predicted to be non-irritant to ocular tissue when topically treated at ≤3% w/v dose.

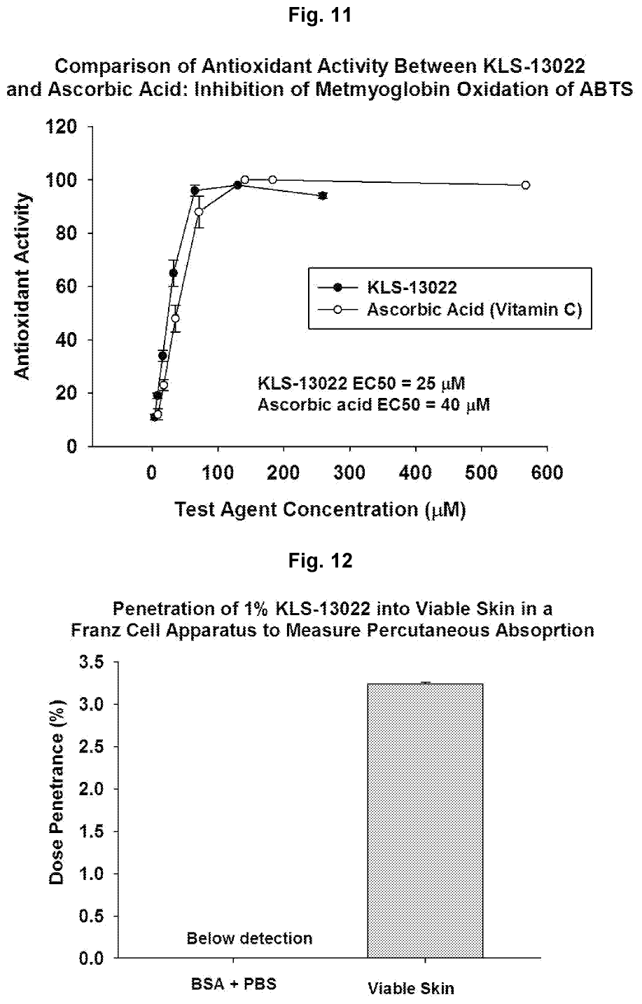

FIG. 11 shows the determination of the antioxidant capacity of KLS-13022. The purpose of this study was to evaluate the overall antioxidant capacity of KLS-13022 for the ability to inhibit the oxidation of ABTS. (2,2′-Azino-di-[3-ethylbenzthiazoline sulphonate]) to ABTS.⁺ by metmyoglobin. Ascorbic Acid (Vitamin C) was used as positive antioxidant control in the assay. Effective antioxidant inhibition activity based on the change of absorbance of blank (vehicle-only) sample (no material added). Results produced by optical density changes for each test material showed that KLS-13022 provided strong antioxidant activity with an average EC₅₀ effective activity of 25.6 μM compared to Ascorbic Acid (EC₅₀=39.7 μM).

FIG. 12 shows a Franz cell dermal penetration of formulation containing KLS-13022. The purpose of this study was to evaluate the percutaneous absorption activity of 1:1 ethanol:water formulation containing 1% w/v of KLS-13022 when topically applied to porcine skin using the Franz cell assay method. Results show that KLS-13022 material penetrated through the viable skin (0.324% in epidermis/dermis) but not into the receptor solution when applied topically in 1:1 ethanol:water formulation. Based on these data, this formulation promoted dermal delivery of KLS-13022 into viable skin, but the compound was predicted to be locally restricted after application by this model.

While the invention has been described in detail and with reference to specific examples thereof, it will be apparent to one skilled in the art that various changes and modifications can be made therein without departing from the spirit and scope thereof.

### REFERENCES CITED

Amber et al. “The Use of Antioxidants in Radiotherapy-Induced Skin Toxicity.” Integrative Cancer Therapies, January 2014, pp. 38-45, doi:10.1177/1534735413490235.
Brenneman D E, Petkanas D, Kinney W A. Pharmacological comparisons between cannabidiol and KLS-13019. (2018) 66: 121-134.
Brenneman D E, Kinney W A, Ward S J. Knockdown siRNA targeting the mitochondrial sodium-calcium exchanger-1 inhibits the protective effect of two cannabinoids against acute paclitaxel toxicity. (2019) 68: 603-619.
Feldmeyer L, Keller M, Niklaus G, Hohl D, Werner S, Beer H-D. The inflammasome mediates UVB-induced activation and secretion of interleukin-1b by Kertinocytes. (2007) Current Biol. 17: 1140-1145.
Kammeyer, A., & Luiten, R. M. (2015). Oxidation events and skin aging. Ageing research reviews, 21, 16-29.
Kinney W, McDonnell M, Zhong H, Liu C, Yang L, Ling W, Qian T, Chen Y, Cai Z, Petkanas D, Brenneman D E. (2016) Discovery of KLS-13019, a Cannabidiol-Derived Neuroprotective Agent, with Improved Potency, Safety, and Permeability. ACS Medicinal Chemistry Letters. 7:424-428.
Perraud A-L, Rao D M, Kosmacek E A, Dagunts A, Oberley-Deegan R E, Gally F. The ion channel, TRPM2, contributes to the pathogenesis of radiodermatitis. (2019) Rad Environ. Biophys 58: 89-98.
Schmidt M, Raghavan B, Muller V, Vogl T, Fejer G, Tchaptchet S. Keck S, Kalis C, Nielsen P J, Galanos C, Roth J, Skerra A, Martin S F, Freudenberg M A, Goebleler M. Crucial role for human Toll-like receptor 4 in the development of contact allergy to nickel. (2010) Nat Immunol 11: 814-819.
U.S. Pat. No. 9,611,213 B2.
U.S. Ser. No. 10/004,722 B2.

What is claimed is:

1. A functionalized 1,3-benzene diol selected from the group consisting of 5-(2-(1H-1,2,3-triazol-1-yl)ethyl)-2-((1R,6R)-3-methyl-6-(prop-1-en-2-yl)cyclohex-2-enyl)benzene-1,3-diol and ethyl 3-(3,5-dihydroxy-4-((1R,6R)-3-methyl-6-(prop-1-en-2-yl)cyclohex-2-enyl)benzyl)azetidine-1-carboxylate.

2. A method of treating or preventing a dermatological disorder, said method comprising applying to skin a therapeutically effective amount of a functionalized 1,3-benzene diol selected from the group consisting of:
formula (I): 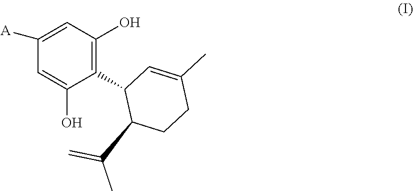
including hydrates, solvates, pharmaceutically acceptable salts, prodrugs and complexes thereof, wherein:
A is selected from the group consisting of 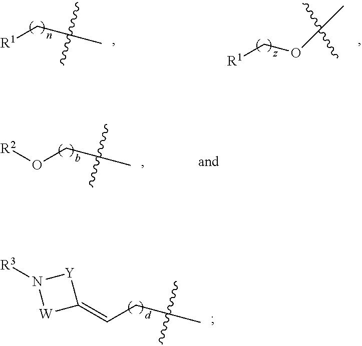
z is 0, 1, or 2;
when A is 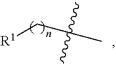
R¹ is selected from the group consisting of 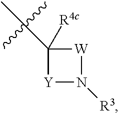
when A is 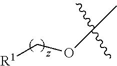
and z is 0, R¹ is 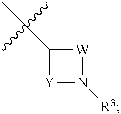
when A is 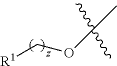
and z is 1, R¹ is 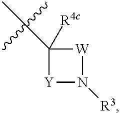
when A is 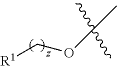
and z is 2, R¹ is selected from the group consisting of 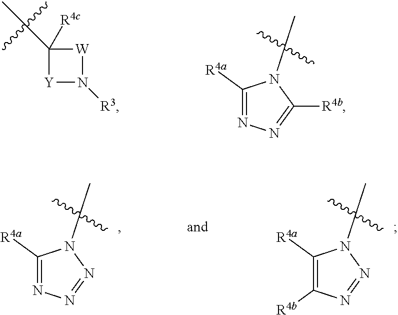
when R¹ is 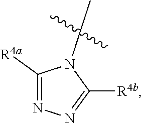
n is not 0;
when R¹ is 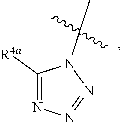
n is not 0;
when R¹ is 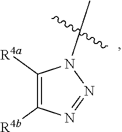
n is not 0;
R² is 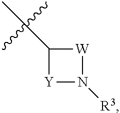
W is (CH₂)ₘ;
m is 1 or 2;
Y is (CH₂)_{q};
q is 1 or 2;
n is 0, 1, 2, or 3;
b is 0, 1, 2, or 3;
d is 0, 1, 2, or 3;
R³ is selected from the group consisting of COR⁵, CO₂R⁶, CONR⁷R⁷, SO₂NR^{7a}R⁷, SO₂R⁸, and optionally substituted heteroaryl;
R^{4a} and R^{4b} are each independently selected from the group consisting of hydrogen and C₁₋₆ alkyl;
R^{4c} is selected from the group consisting of hydrogen and OH;
R⁵ is selected from the group consisting of hydrogen, unsubstituted C₁₋₆ alkyl, substituted C₁₋₆ alkyl, unsubstituted heteroaryl, substituted heteroaryl, —C(R^{9a}R¹⁰)NR^{7a}R⁷, and —C(R^{9a}R^{9b})OR¹⁰;
R⁶ is unsubstituted C₁₋₆ alkyl or substituted C₁₋₆ alkyl;
R^{7a} and R^{7b} are each independently selected from the group consisting of hydrogen, unsubstituted C₁₋₆ alkyl and substituted C₁₋₆ alkyl;
R⁸ is selected from the group consisting of hydrogen, unsubstituted C₁₋₆ alkyl, substituted C₁₋₆ alkyl, unsubstituted heteroaryl and substituted heteroaryl;
R^{9a} and R^{9b} are each independently selected from the group consisting of hydrogen, C₁₋₆ alkyl, C₃₋₇ branched alkyl, CH₂OH, CH(OH)CH₃, CH₂Ph, CH₂(4-OH-Ph), (CH₂)₄NH₂, (CH₂)₃NHC(NH₂)NH, CH₂(3-indole), CH₂(5-imidazole), CH₂CO₂H, CH₂CH₂CO₂H, CH₂CONH₂, and CH₂CH₂CONH₂; and
R¹⁰ is selected from the group consisting of hydrogen and C₁₋₆ alkyl;
formula (II) 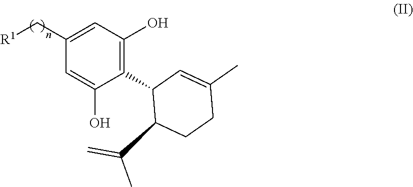
including hydrates, solvates, pharmaceutically acceptable salts, and complexes thereof, wherein R¹ and n of formula (II) are as defined above with respect to formula (I);
formula (III): 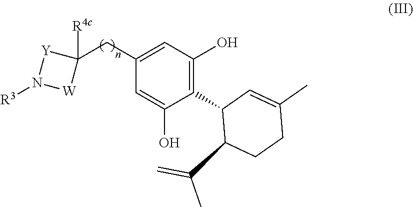
including hydrates, solvates, pharmaceutically acceptable salts, and complexes thereof, wherein R³, R^{4c}, Y, W, and n of formula (III) are as defined above with respect to formula (I);
formula (IV): 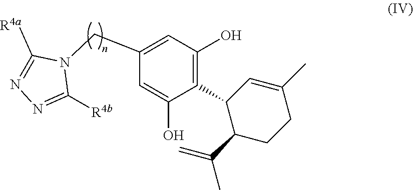
including hydrates, solvates, pharmaceutically acceptable salts, and complexes thereof, wherein n, R^{4a} and R^{4b} are as defined above with respect to formula (I);
formula (V): 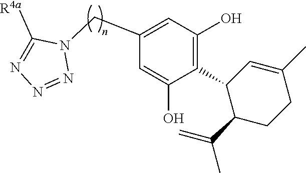
including hydrates, solvates, pharmaceutically acceptable salts, and complexes thereof, wherein n and R^{4a} are as defined above with respect to formula (I);
formula (VI): 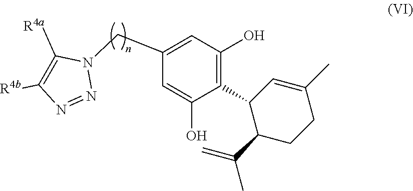
including hydrates, solvates, pharmaceutically acceptable salts, and complexes thereof, wherein n, R^{4a} and R^{4b} are as defined above with respect to formula (I);
formula (VII): 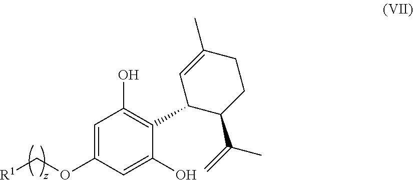
including hydrates, solvates, pharmaceutically acceptable salts, and complexes thereof, wherein R¹ and z are as defined above with respect to formula (I);
formula (VIII): 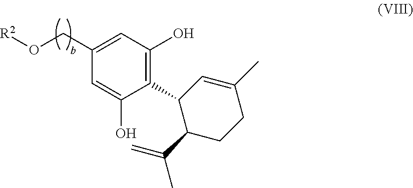
including hydrates, solvates, pharmaceutically acceptable salts, and complexes thereof, wherein R² and b are as defined above with respect to formula (I);
formula (IX): 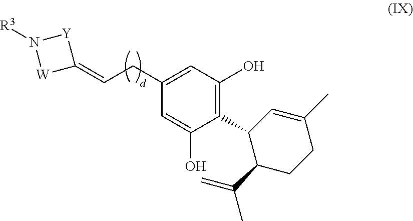
including hydrates, solvates, pharmaceutically acceptable salts, and complexes thereof, wherein R³, Y, W, and d of formula (IX) are as defined above with respect to formula (I); and
formula (X): 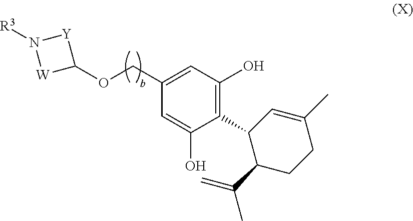
including hydrates, solvates, pharmaceutically acceptable salts, and complexes thereof, wherein R³, Y, W, and b of formula (X) are as defined above with respect to formula (I).

3. The method of claim 2, wherein the dermatological disorder is radiation dermatitis or allergic contact dermatitis.

4. The method of claim 2 or 3, wherein the functionalized 1,3-benzene diol is 5-(2-(1H-1,2,3-triazol-1-yl)ethyl)-2-((1R,6R)-3-methyl-6-(prop-1-en-2-yl)cyclohex-2-enyl)benzene-1,3-diol.

5. The method of any one of claims 2-4, wherein the functionalized 1,3-benzene diol produces an analgesic effect.

6. The method of any one of claims 2-5, wherein the functionalized 1,3-benzene diol exhibits therapeutic effects by locally restricted tissue actions that do not involve significant entry in systemic circulation.

7. The method of any one of claims 2-6, further comprising applying to the skin an additional agent therapeutically effective to treat irradiated skin in a formulation that both solubilizes and enhances protection against oxidative damage in skin tissue.

8. The method of any one of claims 2-7, further comprising applying soy lecithin to the skin.

9. The method of claim 8, wherein the soy lecithin comprises phosphatidylcholine with a polyunsaturation of the fatty acyl composition of at least 63%.

10. The method of any one of claims 2-9, wherein the method inhibits a release of inflammatory cytokines in skin tissue produced by irradiation.

11. The method of any one of claims 2-10, wherein the functionalized 1,3-benzene diol is applied to the skin at a concentration less than 3% (wt/vol) which does not produce skin cell irritancy at therapeutic doses.

12. A method of treating or preventing ocular irritation, said method comprising applying to ocular tissue a therapeutically effective amount of a functionalized 1,3-benzene diol, wherein the therapeutically effective amount does not produce ocular irritancy.

13. The method of claim 12, wherein the functionalized 1,3-benzene diol is 5-(2-(1H-1,2,3-triazol-1-yl)ethyl)-2-((1R,6R)-3-methyl-6-(prop-1-en-2-yl)cyclohex-2-enyl)benzene-1,3-diol.

14. A functionalized 1,3-benzene diol for use in treating or preventing a dermatological disorder or an ocular irritation, said functionalized 1,3-benzene diol being selected from the group consisting of:
formula (I): 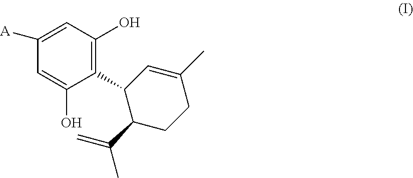
including hydrates, solvates, pharmaceutically acceptable salts, prodrugs and complexes thereof, wherein:
A is selected from the group consisting of 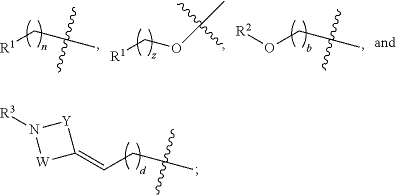
z is 0, 1, or 2;
when A is 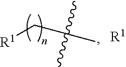
is selected from the group consisting of 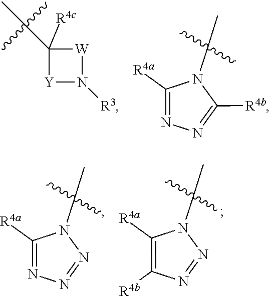
when A is 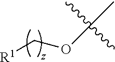
and z is 0, R¹ is 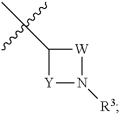
when A is 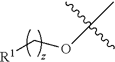
and z is 1, R¹ is 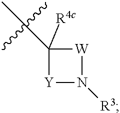
when A is 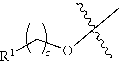
and z is 2, R¹ is selected from the group consisting of 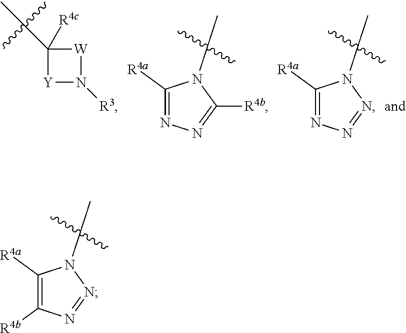
when R¹ is 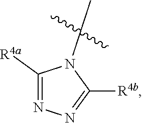
n is not 0;
when R¹ is 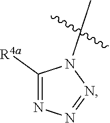
n is not 0;
when R¹ is 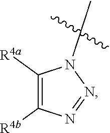
n is not 0;
R² is 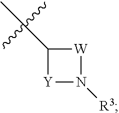
W is (CH₂)ₘ;
m is 1 or 2;
Y is (CH₂)_{q};
q is 1 or 2;
n is 0, 1, 2, or 3;
b is 0, 1, 2, or 3;
d is 0, 1, 2, or 3;
R³ is selected from the group consisting of COR⁵, CO₂R⁶, CONR^{7a}R^{7b}, SO₂NR^{7a}R^{7b}, SO₂R⁸, and optionally substituted heteroaryl;
R^{4a} and R^{4b} are each independently selected from the group consisting of hydrogen and C₁₋₆ alkyl;
R^{4c} is selected from the group consisting of hydrogen and OH;
R⁵ is selected from the group consisting of hydrogen, unsubstituted C₁₋₆ alkyl, substituted C₁₋₆ alkyl, unsubstituted heteroaryl, substituted heteroaryl, —C(R^{9a}R^{9b})NR^{7a}R^{7b}, and —C(R^{9a}R^{9b})OR¹⁰;
R⁶ is unsubstituted C₁₋₆ alkyl or substituted C₁₋₆ alkyl;
R^{7a} and R^{7b} are each independently selected from the group consisting of hydrogen, unsubstituted C₁₋₆ alkyl and substituted C₁₋₆ alkyl;
R⁸ is selected from the group consisting of hydrogen, unsubstituted C₁₋₆ alkyl, substituted C₁₋₆ alkyl, unsubstituted heteroaryl and substituted heteroaryl;
R^{9a} and R^{9b} are each independently selected from the group consisting of hydrogen, C₁₋₆ alkyl, C₃₋₇ branched alkyl, CH₂OH, CH(OH)CH₃, CH₂Ph, CH₂(4-OH-Ph), (CH₂)₄NH₂, (CH₂)₃NHC(NH₂)NH, CH₂(3-indole), CH₂(5-imidazole), CH₂CO₂H, CH₂CH₂CO₂H, CH₂CONH₂, and CH₂CH₂CONH₂; and
R¹⁰ is selected from the group consisting of hydrogen and C₁₋₆ alkyl;
formula (II) 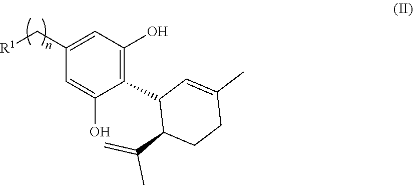
including hydrates, solvates, pharmaceutically acceptable salts, and complexes thereof, wherein R¹ and n of formula (II) are as defined above with respect to formula (I);
formula (III): 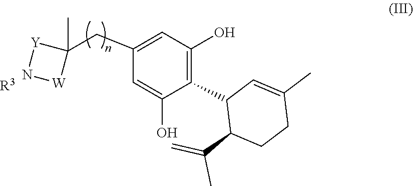
including hydrates, solvates, pharmaceutically acceptable salts, and complexes thereof, wherein R³, R^{4c}, Y, W, and n of formula (III) are as defined above with respect to formula (I);
formula (IV): 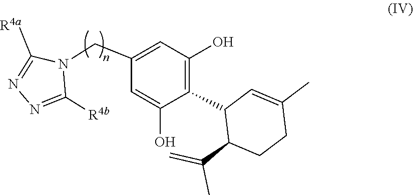
including hydrates, solvates, pharmaceutically acceptable salts, and complexes thereof, wherein n, R^{4a} and R^{4b} are as defined above with respect to formula (I);
formula (V): 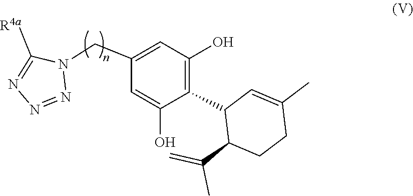
including hydrates, solvates, pharmaceutically acceptable salts, and complexes thereof, wherein n and R^{4a} are as defined above with respect to formula (I);
formula (VI): 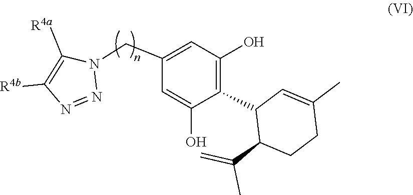
including hydrates, solvates, pharmaceutically acceptable salts, and complexes thereof, wherein n, R^{4a} and R^{4b} are as defined above with respect to formula (I);
formula (VII): 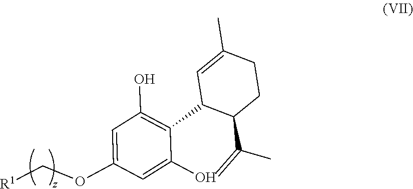
including hydrates, solvates, pharmaceutically acceptable salts, and complexes thereof, wherein R¹ and z are as defined above with respect to formula (I);
formula (VIII): 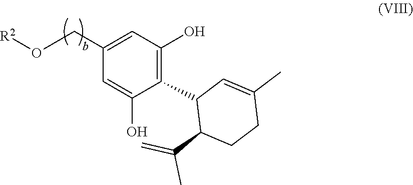
including hydrates, solvates, pharmaceutically acceptable salts, and complexes thereof, wherein R² and b are as defined above with respect to formula (I);
formula (IX): 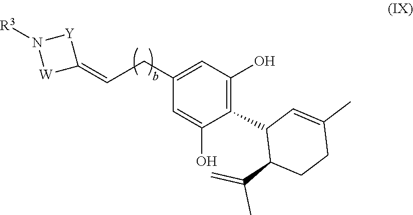
including hydrates, solvates, pharmaceutically acceptable salts, and complexes thereof, wherein R³, Y, W, and d of formula (IX) are as defined above with respect to formula (I); and
formula (X): 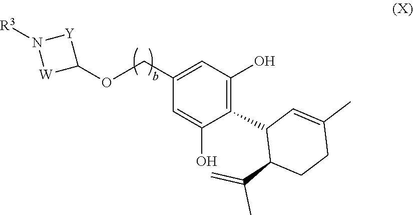
including hydrates, solvates, pharmaceutically acceptable salts, and complexes thereof, wherein R³, Y, W, and b of formula (X) are as defined above with respect to formula (I).

15. The functionalized 1,3-benzene diol of claim 14, which is selected from the group consisting of 5-(2-(1H-1,2,3-triazol-1-yl)ethyl)-2-((1R,6R)-3-methyl-6-(prop-1-en-2-yl)cyclohex-2-enyl)benzene-1,3-diol and ethyl 3-(3,5-dihydroxy-4-((1R,6R)-3-methyl-6-(prop-1-en-2-yl)cyclohex-2-enyl)benzyl)azetidine-1-carboxylate.
